(19) United States
(12) Patent Application Publication
(10) Pub. No.: US 2012/0101089 A1
(43) Pub. Date: Apr. 26, 2012

(54) Anti-Emetic Substance

(76) Inventors: Ashwani Agarwal, Dallas, TX (US); Ankit Bharat, St. Louis, MO (US)

(21) Appl. No.: 13/156,133

(22) Filed: Jun. 8, 2011

Related U.S. Application Data
(60) Provisional application No. 61/406,665, filed on Oct. 26, 2010.

(51) Int. Cl. A61K 31/5415; A61P 1/08

(52) U.S. Cl. 514/226.2; 514/224.8

(57) ABSTRACT

The present invention provides a therapeutic solution for effective control of symptoms related to nausea and vomiting. The therapeutic solution is a pharmaceutical composition which combines anti-emetics of different classes. These classes include dopamine receptor antagonists, serotonin receptor antagonists, butyrphenones, and neurokinin receptor antagonists. The combination of different anti-emetics mitigates adverse affects of a single anti-emetic alone while increasing drug efficacy and decreasing cost of administration to the individual patient.

### CROSS REFERENCE TO RELATED APPLICATIONS

This application claims the benefit of provisional Application No. 61/406,665, which was filed on Oct. 26, 2010.

### TECHNICAL FIELD OF THE INVENTION

The present invention relates generally to field of anti-emetics and specifically to a therapeutic strategy of combining drugs with anti-emetic properties into a single pill, liquid or transdermal for ease of administration and more effective control of symptoms related to nausea and vomiting.

### BACKGROUND OF THE INVENTION

Nausea and vomiting occur in a variety of settings. For example, nausea and vomiting is extremely common in cancer patients undergoing chemotherapy and in patients undergoing surgery, especially intestinal, neurological, otolaryngological and opthalmological procedures. Additionally, nausea and vomiting are also seen commonly in benign conditions like headaches (especially migraines), under conditions of physiological or mental stress, motion sickness, food intolerance, or gastroenteritis. See Table 1. Hence, a broad spectrum of conditions can lead to nausea and vomiting.

Nausea and vomiting are amongst the most distressing symptoms reported in medical literature. Moreover, they have the potential of causing serious medical consequences including but not limited to electrolyte abnormalities, dehydration, malnutrition, acute renal failure, and cardiac arrythmias. See Table 2. Hence, prompt control of these symptoms is warranted in most circumstances.

Many anti-emetic agents exist to control the symptoms of nausea and vomiting. These agents work on specific neurochemical pathways and thus control symptoms in separate ways. However, each of these agents has adverse effects on the patient which tend to increase with amount of dosage. Therefore, the existence of a strategy that can combine the benefits of separate agents while ameliorating the adverse effects is desirable.

TABLE 1
Common Conditions Associated With Nausea and Vomiting

| | |
|---|---|
| Benign | Motion, sea, or air sickness |
| | Pregnancy |
| Medical conditions | Vestibular diseases |
| | Gastroenteritis |
| | Cancer |
| | Gastric outlet or bowel obstruction |
| | Post-operative ileus |
| | Migraine |
| | Cyclic vomiting syndrome |
| Intervention related | Anesthesia |
| | Post operative without ileus |
| | Chemotherapy |

TABLE 2
Consequences of In-Hospital Nausea and Vomiting

| | |
|---|---|
| Nutrition | Delay in intake of fluid and food |
| Medical | Dehydration |
| | Hypotension |
| | Electrolyte imbalance |
| | Cardiac arrhythmias |
| | Renal failure |
| | Metabolic acidosis or alkalosis |
| | Aspiration pneumonia |
| Surgical | Dehiscence of surgical repair (hernia, |
| | vascular anastomosis, flaps, wound) |
| | Hematoma formation |
| | Increased intracranial pressure |
| | Esophageal tears |
| Financial | Increased medical and nursing costs |
| | Delay in discharge |

### SUMMARY OF THE INVENTION

The present invention provides a therapeutic strategy of combining anti-emetics of different classes into a single pill or other delivery forms for ease of administration, more effective control of symptoms related to nausea and vomiting, improved patient compliance, less adverse effects, and a reduction in cost of treatment.

The single delivery approach improves ease of administration by medical personnel. Since patients with persistent nausea and vomiting have difficulty swallowing, combining multiple agents into one form improves ease of administration.

Also, the single delivery approach improves patient compliance. The invention has a major role in control of symptoms following discharge, and the patient will better comply with treatment when the prescribed medication intake is less onerous to the patient. Hence, the single delivery approach leads to better patient compliance.

Additionally, the single delivery approach ameliorates adverse effects of individual anti-emetic agents. Most anti-emetic agents have significant adverse effects that increase in frequency in higher doses. The invention controls nausea and vomiting with much lower doses of the agents, which decreases the overall incidence of adverse effects.

Furthermore, the invention improves effective control of the symptoms associated with nausea and vomiting. Since each of these ailments has a multifactorial pathogenesis involving multiple neurochemical pathways, multiple agents that individually target different pathways will improve the overall efficacy of treatment.

Moreover, use of a single delivery approach containing multiple agents will be much cheaper than using multiple agents in separate delivery vehicles due to the overall decrease in manufacturing and processing costs. Hence, the invention reduces cost of treatment.

In one embodiment of the present invention, the anti-emetic composition comprises the anti-emetic agents ondansetron and promethazine.

In another embodiment, the anti-emetic composition comprises the anti-emetic agents granisetron and promethazine.

In another embodiment, the anti-emetic composition comprises the anti-emetic agents dolastetron and promethazine.

In yet another embodiment, the anti-emetic composition comprises the anti-emetic agents promethazine and the NK1 receptor antagonist aprepitant.

In yet another embodiment, the anti-emetic composition comprises a NK1 receptor antagonist and a serotonin receptor antagonist.

In yet another embodiment, the anti-emetic composition comprises a NK1 receptor antagonist and an anticholinergic.

In yet another embodiment, the anti-emetic composition comprises a serotonin receptor antagonist and an anticholinergic.

In yet another embodiment, the anti-emetic composition comprises a serotonin receptor antagonist and a butyrophenone.

In yet another embodiment, the anti-emetic composition comprises a NK1 receptor antagonist and a butyrophenone.

In yet another embodiment, the anti-emetic composition comprises a serotonin receptor antagonist and a butyrophenone.

In yet another embodiment, the anti-emetic composition comprises a serotonin receptor antagonist and metoclopromide.

In yet another embodiment, the anti-emetic compositions comprises a NK1 receptor antagonist and metoclopromide.

In yet another embodiment, the anti-emetic compositions comprises a serotonin receptor antagonist and an antihistamine.

In yet another embodiment, the anti-emetic compositions comprises a NK1 receptor antagonist and an antihistamine.

### DETAILED DESCRIPTION OF THE PREFERRED EMBODIMENT

Generally, this invention relates to the therapeutic treatment of patients exhibiting symptoms related to nausea and vomiting. The pharmaceutical composition of the present invention is a combination of anti-emetic agents from different classes into a single formation.

Anti-emetic agent is defined and hereinafter used as a drug which inhibits the symptoms related to nausea and vomiting.

Anti-emetic agents can be categorized into different classes. Classes of anti-emetic agents are the different categories of anti-emetic drugs, each having a specific action on the neurochemical pathways triggered by stimuli that lead to nausea and vomiting.

### Pathophysiology of Nausea and Vomiting

Vomiting (emesis) is the forceful expulsion of gastrointestinal (hereinafter GI) contents through the mouth. Retching is the rhythmic action of respiratory muscles preceding vomiting. Both retching and vomiting are objective patient experiences. Nausea is a subjective personal patient experience which may or may not be associated with vomiting. The vomiting reflex was hypothesized by Borison and Wang to be a complex act that is coordinated by the vomiting centre. (Borison, H L, Wang S C: Physiology and pharmacology of vomiting. Pharmacol Rev 5; 193-230, 1953.) The vomiting centre is located in the lateral reticular formation of the medulla oblongata of the mid-brainstem central nervous system (hereinafter CNS) in close proximity to the nucleus of the solitary tract and area postrema at the level of the dorsal motor nucleus of the vagus nerve. The chemoreceptor trigger zone (hereinafter CTZ) is located in the area postrema near the vomiting centre at the bottom of the fourth ventricle.

The process of nausea, retching and vomiting is coordinated by the vomiting centre. Stimulation can be initiated from the periphery (oropharynx, mediastinum, GI tract, renal pelvis, peritoneum and genitalia) and centrally from the CNS (cerebral cortex, labyrinthine, otic, vestibular apparatus). Stimuli are relayed from the periphery to the vomiting centre by the autonomic nervous system afferent neurons of the vagus nerve. There is afferent input to the area postrema from the glossopharyngeal and vagal nerves. Central cerebral sensory stimuli occur directly and are transmitted by the CTZ, area postrema and nucleus of the solitary tract in the lateral reticular formation of the medulla to the vomiting centre. Chemicals in the cerebro-spinal fluid (hereinafter CSF) and blood have a direct stimulating effect at the vomiting centre. The areas in the CNS associated with balance, vasomotor activity, salivation, respiration and bulbar control are located near, and have innervation to, the vomiting centre. The close proximity of these areas to the vomiting centre corresponds to the physiological reactions often seen with Post-Operative Nausea and Vomiting (hereinafter PONV), such as salivation, increased swallowing, sweating, pallor, tachypnea, tachycardia, cardiac dysrhythmias and motion sickness. The CTZ contains high concentrations of enkephalin, opioid and dopamine D2 receptors. The area postrema has high concentrations of opioid, D2 and serotonin (5-hydroxytryptamine; 5-HT) receptors. The nucleus of the solitary tract has a pre-dominance of enkephalin, histamine, muscarinic and cholinergic receptors. These emetic neuroreceptor areas serve as sensors and are stimulated by drugs, electrolytes and metabolic chemicals, causing impulses to be relayed to the vomiting centre, thereby initiating the vomiting reflex. Blockade of these neurochemical receptor sites is the mechanism of action of the anti-emetic medications commonly used for PONV.

### Consequences of Nausea and Vomiting

The consequences of PONV are patient-, physiological-, medical-, surgical-, anesthesia-, hospital and cost-related. Prolonged vomiting may lead to electrolyte imbalances (hypokalaemia, hypochloraemia, hyponatraemic metabolic alkalosis) and dehydration. Aspiration of gastric contents in the perioperative period is an important anesthesia related concern and consequence of PONV. A Mallory Weis tear, esophageal rupture, wound dehiscence and haematoma formation beneath skin flaps are important postoperative surgical-related concerns that may occur with PONV following abdominal, vascular, eye or plastic surgeries. Patient-related concerns are the postoperative pain and discomfort from PONV. Hospital and increased nursing care time contribute to the economic-related consequences of PONV.

### Classes of Drugs and Mechanism of Action

Currently, several different classes of drugs are available for the treatment of nausea and vomiting. The drugs in each of these classes have a specific action on the neurochemical pathways triggered by stimuli that lead to nausea and vomiting.

### Anticholinergics

Anticholinergic agents are among the oldest first-generation class of anti-emetics. Anticholinergics are potent inhibitors of muscarinic and cholinergic CNS emetic receptors in the cerebral cortex and pons. Compounds with selective muscarinic M3 and M5 receptor antagonism possess activity against motion sickness. As tertiary amines, atropine and scopolamine cross the blood-brain barrier and have efficacy against nausea and vomiting. Scopolamine is an effective preoperative anti-emetic, especially when sedation is required. Atropine has weaker anti-emetic properties than scopolamine. Both appear to be more effective against motion-induced vomiting than motion induced nausea. Prophylactic transdermal scopolamine is available and more effective. Scopolamine blocks impulses from vestibular nuclei to higher areas in the CNS, reticular activating formation and vomiting centre. Scopolamine helps to correct the CNS imbalance of acetylcholine and noradrenaline (norepinephrine) that occurs in patients who have motion sickness. Adding an anticholinergic medication to an opioid for use as premedication has been shown to decrease emesis. Transdermal scopolamine is effective in preventing the post-operative nausea and vomiting caused by opioids (i.e. epidural morphine). The main disadvantages of the use of anticholinergics include anticholinergic adverse effects, which are sedation, blurred vision, mydriasis, dry mouth, memory loss, urinary retention, hallucinations, confusion and disorientation. Anticholinergics can be used in combination with other classes of anti-emetics such as serotonin receptor antagonists, NK1 receptor antagonists, and dopamine receptor antagonists in proportion so as to reduce the toxicity of each compound and to increase overall efficacy.

### Dopamine Receptor Antagonists

The phenothiazines (promethazine, prochlorperazine), benzamides (metaclopramide) and butyrophenones (droperidol) are strong dopamine receptor antagonists (D2 antagonists). Dopamine receptors are a class of metabotropic G protein-coupled receptors that are prominent in the CNS. A receptor antagonist is defined and hereinafter used as a type of drug that does not provoke a biological response itself upon binding to a receptor, but blocks or dampens agonist-mediated responses. Whereas an agonist causes an action, an antagonist blocks the action of the agonist.

### Phenothiazines

Phenothiazines (promethazine, chlorpromazine, prochlorperazine, perphenazine, thiethylperazine) are among the most widely used anti-emetic medications worldwide. The phenothiazines have a common tricyclic nucleus. The attached chemical radical group on the tenth position of the tricyclic nucleus appears to determine anti-emetic efficacy. This side chain radical group may be either aliphatic (promethazine, chlorpromazine) or heterocyclic (prochlorperazine, perphenazine, thiethylperazine). The aliphatic phenothiazines have less anti-emetic potency and more sedative effects than the heterocyclic phenothiazines. The phenothiazines exert a direct D2 receptor blocking effect in the CTZ with moderate antihistaminergic and anticholinergic actions. These medications are used as sedatives and major tranquilizers, and are especially effective in countering the effect of certain drugs (i.e. opioids) on the CTZ. Although they are effective for the prevention and treatment of chemotherapy induced and post-operative nausea and vomiting, they are less effective against motion sickness and have no effect on gastric emptying. Chlorpromazine has PONV anti-emetic effectiveness, with adverse effects of sedation and hypotension, but it is not effective for the prevention of motion sickness. Promethazine is an effective prophylactic anti-emetic with more sedation and a more prolonged recovery period from anesthesia than the heterocyclic phenothiazines (i.e. prochlorperazine). Promethazine is the most effective of the phenothiazines for prevention of motion sickness. The long duration of action of promethazine makes it preferable to scopolamine. Heterocyclic phenothiazines (perphenazine, prochlorperazine) have a piperazine ring substituted at position number 10 of the tricyclic nucleus. Perphenazine and prochlorperazine are the 2 heterocyclic phenothiazines most commonly used as anti-emetics. Perphenazine is useful for the prevention and treatment of PONV caused by opioids. As a prophylactic PONV anti-emetic in paediatric patients, intravenous perphenazine 70 μg/kg decreased emesis after tonsillectomy. This prophylactic dose (70 μg/kg) of perphenazine also was more effective than dexamethasone 150 μg/kg[86] and equally as effective as ondansetron 150 μg/kg in preventing PONV in children after tonsillectomy operations. Prophylactic intramuscular prochlorperazine 0.2 mg/kg and intravenous ondansetron 0.06 mg/kg had similar and better efficacy than intravenous prochlorperazine 0.01 mg/kg in preventing PONV following tympanoplasty. Intramuscular prochlorperazine has an onset time of 30 to 60 minutes and lasts up to 4 hours. Prochlorperazine and perphenazine have similar anti-emetic effectiveness, but perphenazine causes more sedation. Similarly, prochlorperazine and promethazine were also determined to be equally as effective for PONV prevention, but promethazine had more postoperative sedation. Although the heterocyclic phenothiazines are more effective anti-emetic medications than the aliphatic phenothiazines, they have a higher incidence of extrapyramidal symptoms (EPS). These include (i) akathesia (motor restlessness); (ii) acute dystonia (spasmodic contractures producing trismis, torticollis, opisthotonos and oculogyric crisis); (iii) pseudo-parkinsonism; and (iv) tardive dyskinesia. Treatment of these EPS is accomplished by (i) discontinuing the heterocyclic phenothiazine causing the problem; (ii) administering another phenothiazine that does not have the heterocyclic ring (i.e. aliphatic, promethazine); (iii) switching to a different class of anti-emetics; or (iv) administering diphenhydramine. EPS are less common when the heterocyclic phenothiazines are administered in combination with opiates. The neuroleptic malignant syndrome (hypyrexia, muscle rigidity, autonomic instability, and altered mental status) has been reported with the phenothiazines, droperidol and metoclopramide. Anticholinergic adverse effects of the phenothiazines include dry mouth, urinary retention, tachycardia and drowsiness. The hypotensive adverse effects can be treated with intravenous fluid hydration and phenylephrine.

### Butyrophenones

The butyrophenones (haloperidol, droperidol) have a similar pharmacological and anti-emetic effectiveness profile as the phenothiazines. They are α-blockers, with adverse effects of sedation and EPS. They are also strong D2 receptor antagonists that act at the CTZ and area postrema. Both haloperidol and droperidol are effective anti-emetic medications for the prevention and treatment of PONV. However, droperidol is more commonly used in anesthesia than haloperidol. Intramuscular haloperidol has an onset of action of approximately 30 minutes and duration of approximately 12 hours. Haloperidol 7 μg/kg intravenously, and 0.5 to 4.0 mg intramuscularly for prevention and 1 mg intramuscularly for treatment, has been shown to be effective in PONV, with little sedative effect. Haloperidol causes less sedation than prochlorperazine. Droperidol is similar to haloperidol and the phenothiazines in regard to effectiveness for PONV prevention and treatment. Intramuscular droperidol 5 mg has equivalent anti-emetic effectiveness to intramuscular haloperidol 2 mg. The onset of anti-emetic action for droperidol is slower than that of haloperidol or prochlorperazine. The effect of droperidol is longer (as long as 24 hours following administration), even though it has a shorter halflife than haloperidol. Whereas haloperidol appears to act at the D2 receptors in the CTZ and area postrema vomiting centres more rapidly than droperidol, droperidol appears to have a stronger binding affinity for these emetic receptors and is retained at the receptor sites for a longer period of time. When administered immediately before the end of anesthesia, intravenous droperidol 1.25 mg was determined to be superior to intravenous metoclopramide 10 mg, intravenous domperidone 5 mg and an intravenous saline placebo for the prevention of PONV after a balanced (N20/02/opioid) general anesthesia in day-stay gynaecological and major gynaecological surgery. Droperidol 1.25 mg intravenously administered before the end of general anesthesia was also determined to be significantly superior to intravenous metoclopromide 10 mg and a saline placebo in the prevention of PONV in female patients undergoing elective orthopaedic surgery. Droperidol 5 μg/kg intravenously administered 1 hour before the end of anesthesia was effective in preventing PONV in children 11 to 15 years old. Intravenous droperidol 0.625 mg was found to be as effective as intravenous droperidol 1.25 mg for PONV prevention when these doses were administered immediately following intubation. At these doses, droperidol was judged to have few adverse effects (i.e. dystonic reactions, EPS, sedation). With repeated and high doses, both haloperidol and droperidol may cause EPS, anxiety, restlessness, hypotension and postoperative sedation, especially in young adults and the elderly. These adverse effects appear to be more severe (especially the EPS) than those observed with the phenothiazines. These drugs should be used cautiously in outpatient surgery because these adverse effects may delay discharge from outpatient surgery. As haloperidol has a more rapid onset but shorter duration compared with droperidol, a combination of haloperidol and droperidol may be more effective by providing anti-emetic action of more rapid onset and longer duration than either alone. However, the additive adverse effects (sedation, EPS, dystonias) of both drugs would be a disadvantage. A better approach appears to be the administration of intravenous droperidol 0.625 to 2.5 mg immediately after induction of anesthesia, or 0.625 to 1.25 mg 15 to 30 minutes before the end of surgery. Butryophenones will be used in combinations with 5-HT3 antagonists and NK1 receptor antagonists.

### Benzamides

Metoclopramide and domperidone are specific dopamine D2 antagonists, unrelated to the phenothiazines and without antihistamine properties. Metoclopramide is a procainamide derivative and a benzamide prokinetic agent with dual sites of action, blocking D2 receptors in the periphery (GI tract) and centrally (CTZ and area postrema vomiting centres). Metoclopramide increases lower esophageal sphincter tone and promotes gastric motility, which may prevent the delayed gastric emptying caused by opioid analgesics. The anti-emetic efficacy of metoclopramide has been controversial and varied due to the use of different doses, timing of administration, types of surgery and anaesthetic techniques. Because of its short duration of action (1 to 2 hours), metoclopramide does not appear to be as effective for PONV prevention when administered before anesthesia. To allow an adequate plasma concentration for anti-emetic effectiveness, metoclopramide appears to be best administered either at the end of surgery or after initial entry to the PACU. Metoclopramide appears to have better anti-emetic efficacy in the immediate postoperative period when administered to patients receiving opioids for postoperative pain. In this way, the prokinetic effects of metoclopramide improve motility of the stomach and small bowel, and counteract the delayed stomach emptying effect of opioids. Preoperative metoclopramide 10 and 20 mg intramuscularly abolished the pre-anaesthetic emetic effects of pethidine. An additional 10 to 20 mg of metoclopramide intramuscularly administered at the end of the operation reduced the emetic effects of pethidine, but had less effect when morphine was given. Metoclopramide 10 mg intravenously has been shown to be as effective as intravenous droperidol 1.25 mg and more effective than intravenous propofol 10 mg for the treatment of PONV. In addition, metoclopramide 15 mg and 0.15 mg/kg administered intravenously (immediately after the umbilical cord was clamped) has been shown to effectively reduce the incidence of nausea and vomiting during epidural anesthesia (lidocaine, morphine) for elective Caesarean section. Metoclopramide has relatively few adverse effects when used in low doses and does not affect perioperative haemodynamic stability or anaesthetic recovery time. As with droperidol and haloperidol, EPS have occurred following metoclopramide use. Domperidone is a benzimidazole medication pharmacologically similar (but with a different chemical structure) to metaclopramide. Domperidone is a D2 antagonist acting at the CTZ. Domperidone has a lower incidence of EPS than metoclopramide. Similar to metaclopramide, domperidone has prokinetic properties that increase gastric emptying in combinationwith lower oesophageal sphincter tone. While domperidone appears to have similar effectiveness to metaclopramide for the prevention of PONV, it appears to be more effective than metoclopramide for the treatment of active PONV. Domperidone 4 and 10 mg intravenously has been shown to be superior to intravenous metaclopramide 10 mg when used for the treatment of PONV. Metoclopramide can be combined with longer acting serotonin receptor antagonists and NK1 receptor antagonists for maximum efficacy since metocopramide is short-acting.

Benzquinamide is a short-acting benzquinalone derivative that has anti-emetic efficacy secondary to its antihistamine, anticholinergic and antiserotonin properties. The anti-emetic action of benzquinamide occurs via blockade of emetic receptors in the CTZ. Sedation is a common adverse effect. Intramuscular benzquinamide has been found to be effective for the prevention and treatment of PONV. Benzquinamide should not be administered intravenously because of its tendency to cause tachycardia, hypertension and ventricular arrhythmias.

### Antihistamines

Antihistamines (dimenhydrinate, diphenhydramine, cyclizine, hydroxyzine) act by blocking (i) acetylcholine in the vestibular apparatus; and (ii) histamine H1 receptors in the nucleus of the solitary tract. Antihistamines are effective for the treatment of vertigo and motion sickness. Their main site of action is the vomiting centre and vestibular pathways, with little action at the CTZ. They are the drugs of choice to control PONV following operations on the middle ear, which involve components of the vestibular nerve. Their major disadvantages are sedation, dry mouth, blurred vision, urinary retention, and prolonged anesthesia and PACU recovery times. Cyclizine has similar effectiveness to promethazine in preventing and treating PONV (caused by opioids) and motion sickness. While the overall incidence of adverse effects is less frequent with cyclizine compared with the phenothiazine anti-emetics, excess sedation is the most frequent adverse effect of cyclizine. Hydroxyzine is an anxiolytic anti-emetic medication with antihistamine, anticholinergic and bronchodilatory effects that is useful in treating vertigo, motion sickness and PONV. Hydroxyzine has a duration of action of 4 to 6 hours with minimal circulatory and/or respiratory depression. The sedation and anti-sialogogue anti-emetic effects of hydroxyzine make it a good premedication when given in combination with opioids to supplement their analgesic effect. As hydroxyzine potentiates the CNS depressant action of opioids and barbiturates, the dosage of these medications should be reduced by 50% (or more) when administered concurrently with hydroxyzine. Intramuscular hydroxyzine 100 mg administered after induction of anesthesia (in non-premedicated patients) was shown to decrease the incidence of PONV more effectively than intramuscular droperidol 2.5 mg. Antihistamines can be combined with 5-HT3 antagonists or NK1 receptor antagonists.

### Benzodiazepines

Benzodiazepines (diazepam, midazolam, lorazepam) have sedative, anxiolytic and amnestic properties. They decrease the anxiety and restlessness associated with anesthesia and surgery, thereby decreasing PONV. For children, premedication with benzodiazepines appears to have an anti-emetic effect. Midazolam 75 μg/kg administered intravenously after induction of anesthesia has been found to be effective for the prevention of vomiting after tonsillectomy operations in children. Intravenous lorazepam 10 μg/kg was compared with intravenous droperidol 75 μg/kg in children and administered prophylactically after an inhalation induction, but before the start of strabismus surgery. Lorazepam produced similar anti-emetic effectiveness but less postoperative agitation compared with droperidol. The benzodiazepines do not appear to show true anti-emetic receptor binding affinity, but decrease the production of catecholamines, thereby decreasing anxiety. Their amnesia producing effects are helpful to prevent memory of any existing PONV.

### Serotonin Receptor Antagonists

The serotonin 5-HT3 receptor is highly specific and selective for nausea and vomiting. The 5-HT3 receptor antagonists have been determined to be effective for chemotherapy- and radiation therapy—induced nausea and vomiting. These positive results initiated investigations for their use in PONV.

### Ondansetron

Ondansetron was the first 5-HT3 receptor antagonist evaluated and approved for PONV by both oral and intravenous administration in adults and children. Several studies have been conducted in adults to determine dosage regimens for ondansetron. The optimal effective dose was found to be 8 mg orally administered 1 to 2 hours before anesthesia or 4 mg intravenously at the start of anesthesia. In children older than 2 years, 0.1 mg/kg orally for prevention and 0.1 mg/kg (<40 kg) and 4 mg (>40 kg) intravenously for treatment were determined to be the optimal ondansetron doses for PONV. In these PONV prevention studies, intravenous ondansetron was administered before the start of anesthesia; however, two studies have investigated the efficacy of ondansetron administered at the end of surgery. Both studies determined that the efficacy of intravenous ondansetron 4 mg was significantly better when administered at the end of surgery rather than before induction of anesthesia. This improved efficacy of intravenous ondansetron given at the end of surgery was believed to be related to the length of surgery (with longer operations having decreased ondansetron effectiveness than when administered at the start of surgery). Because of the increased cost of the 5-HT3 receptor antagonists compared with traditional anti-emetics, Kovac and colleagues conducted a multicentre study to compare repeat intravenous administration of ondansetron 4 mg with placebo for the treatment of PONV in patients for whom prophylactic preoperative intravenous ondansetron 4 mg was inadequate (Kovac, A. L., Eberhart, L., Kotarski, J., Clerici, G., Alpel, C., The Palonosetron 04-07 Study Group, (2008). A Randomized, Double-Blind study to evaluate the Efficacy and Safety of Three Different Doses of Palonosetron Versus Placebo in preventing postoperative Nausea and Vomiting Over a 72-hour Period. Anesth. Analg. 107:439-444). Kovac and colleagues determined that, in patients for whom preoperative prophylaxis with intravenous ondansetron 4 mg is not successful, a repeat dose of intravenous ondansetron 4 mg in the post anesthesia care unit (hereinafter PACU) does not appear to offer additional control of PONV. However, the administration of an additional dose of ondansetron 4 mg postoperatively did not result in an increased incidence of adverse events.

### Granisetron

The effectiveness of intravenous granisetron for prevention of PONV has been determined. Dose-ranging studies comparing intravenous granisetron 0.1, 1 and 3 mg in adults, determined that granisetron 1 mg was the optimum effective prophylactic dose when administered immediately before the start of anesthesia for treatment of PONV. However, in other studies on PONV prevention, intravenous granisetron 40 μg/kg was determined to be the optimal dose for adults and children. As is commonly know to those skilled in the art, granisetron and granesitron hydrochloride are the same drug.

### Tropisetron

Tropisetron has been studied for prevention and treatment of PONV in adults and has an elimination half-life of 8 to 12 hours. Tropisetron 5 mg intravenously before the start of anesthesia has been found effective for prevention of PONV after breast and gynaecological surgery. Alon and colleagues determined that intravenous tropisetron 2 mg was the optimal effective dose for the treatment of PONV following a variety of abdominal and non-abdominal surgeries. (Alon and Colleagues. Tropisetron treating established PONV. A & A, March 1998 vol. 86. no. 3; 617-623.)

### Dolasetron

The parent compound, dolasetron, is converted to the active metabolite, hydrodolasetron, by the plasma enzyme carbonyl reductase. Dolasetron has an elimination half-life of 9 minutes and is undetectable in the serum 2 to 4 hours after intravenous administration. Hydrodolasetron has a mean half-life of approximately 7.1 and 8.3 hours for the oral and intravenous forms, respectively, and is responsible for the majority (87%) of the anti-emetic effect. In adults, intravenous dolasetron has been evaluated for PONV prevention and treatment, and the oral formulation for PONV prevention. The recommended intravenous dose of dolasetron for prophylaxis and treatment of PONV in adults is 12.5 mg. The prophylactic dose should be given 15 to 30 minutes before the end of anesthesia. Dolasetron pharmacokinetic data in children indicate that oral doses administered 1 to 2 hours before anesthesia were similar to intravenous doses administered at induction of anesthesia. Dolasetron was determined to have an increased clearance and a shorter half-life in children compared with young healthy adult volunteers. The recommended oral dolasetron dose for prevention of PONV is shown in Table 7, below. Following a model used for chemotherapy induced nausea and vomiting, Kovac et al. studied the utilization of hospital resources in adult patients treated for PONV with intravenous dolasetron. (Kovac Ala. Prevention and treatment of postoperative nausea and vomiting. Drugs 2000; 59:213-243). Treatment with dolasetron was found to significantly decrease the utilization of emesis supplies and other hospital resources, including staff supplies and patient/bed linens. In addition, patients receiving dolasetron used fewer healthcare resources in time spent by hospital personnel than patients who were not treated with dolasetron.

### Corticosteroids

Corticosteroids have been evaluated for their usefulness in preventing PONV after they were found to be effective in preventing chemotherapy-induced nausea and vomiting. An anti-inflammatory and/or membrane stabilising effect may play a role in the anti-emetic action of corticosteroids. Aasboe et al. concluded that intramuscular betamethasone 12 mg, when given before general anesthesia for ambulatory foot or haemorrhoid operations, produced less PONV and postoperative pain in the first 24 hours following surgery. (Aasboe, V, Raeder, J. C. and Groegaard, B. A & A, August 1998 vol. 87 no 2: 319-323.) Intravenous dexamethasone 1 mg/kg significantly decreased the incidence of PONV in children (age 2 to 12 years) undergoing tonsillectomy when administered after a mask inhalation induction and before the start of surgery compared with a saline placebo.

### NK1 Inhibitors (NK1 Receptor Antagonists)

### Aprepitant, Fosaprepitant, Casopitant

NK1 inhibitors are a class of drugs that block the NK1 receptor sites abundant in the nucleus tractis solitaires of the mid brain and the vagal afferent pathways located in the enterchromafin cells of the gut. As is commonly known to those skilled in the art, the terms “NK1 inhibitor” and “NK1 receptor antagonist” are equivalent.

The tachykinin pathway is one of the many pathways implicated for chemo induced nausea and vomitting and is mediated by substance P. Substance P is a neurotransmitter that acts on NK1 receptors, both peripheral and central.

Two multicenter, parallel, double-blind studies using a HEC regimen (cisplatin >50 mg/m²) evaluated the efficacy of aprepitant in combination with ondansetron and dexamethasone compared with standard 5-HT₃ and dexamethasone (Higa G M, Auber M, Altaha R, et al. Concordance between substance P levels and antiemetic guidelines. J Support Oncol. 2009; 7:138-142. Trigg M E, Higa G M. Chemotherapy-induced nausea and vomiting: anti-emetic trials that impact clinical practice. J Oncol Pharm Practice. Jan. 19, 2010.). Patients (n=1105) were evaluated for acute and delayed CINV.

### Miscellaneous

### Ephedrine

In a prospective intramuscular PONV prevention study for outpatient gynecological laparoscopy comparing ephedrine 0.5 mg/kg with droperidol 0.04 mg/kg or saline placebo, ephedrine was determined to have similar anti-emetic effectiveness to droperidol and significantly better effectiveness than placebo without sedative adverse effects or effect on BP. Another study investigated the effect of ephedrine 0.5 mg/kg intramuscularly compared with intravenous propofol 0.25 mg/kg for preventing PONV after laparoscopic surgery. Both the ephedrine and propofol groups were more effective, with no haemodynamic changes, than placebo. Intramuscular ephedrine appears to be an effective alternative anti-emetic for PONV, especially when the PONV may be related to fluid dehydration and orthostatic hypotension.

### Propofol

Since the introduction of propofol as a hypnotic induction agent for outpatient anesthesia, patients administered propofol have appeared to have less PONV. The exact mechanism of the anti-emetic effect of propofol is not known. A recent study determined that the anti-emetic properties of subhypnotic doses of propofol (1 mg/kg/hour) were not related to any antidopaminergic properties. Intraoperative intravenous propofol was found to be equally as effective as intravenous ondansetron 4 mg in preventing PONV during the first 6 hours postoperatively. Subhypnotic intravenous doses of thiopentone versus propofol have been compared for anti-emetic effectiveness at the end of outpatient middle ear surgery. Intravenous propofol administered at a subhypnotic dose of 0.5 mg/kg provided significantly better PONV prophylaxis against retching and vomiting for the first 6 hours after middle ear surgery than thiopentone 1.0 mg/kg. A subhypnotic dose of propofol 0.5 mg/kg intravenously was more effective in preventing PONV after sevoflurane anesthesia than desflurane anesthesia for outpatient laparoscopic cholecystectomy. Another study comparing a 20-hour postoperative 0.1 ml/kg/hour infusion of either propofol or 10% Intralipid (placebo control) determined that the overall occurrence of PONV was less with propofol. A plasma propofol concentration of 343 ng/L achieved with a 10 mg intravenous bolus followed by an infusion of 10 μg/kg/min was determined to be necessary to obtain a 50% reduction in postoperative nausea. However, in another study, an intravenous bolus of propofol 0.1 mg/kg followed by a constant infusion of 1 mg/kg/hour had no effect on PONV. It was hypothesized that this dose was below the PONV efficacy threshold for propofol.

### Efficacy of Different Anti-Emetics

Studies have compared the anti-emetic efficacy of the 5-HT3 receptor antagonists with the older, traditional anti-emetics (i.e. droperidol, metoclopramide) and with other 5-HT3 receptor antagonists. Various studies have compared droperidol with ondansetron. Alon and Himmelseher evaluated the anti-emetic efficacy of ondansetron with metoclopramide and droperidol and concluded that fewer patients treated with intravenous ondansetron 8 mg prior to anesthesia had emesis than with intravenous metoclopramide 10 mg or droperidol 0.625 mg (Alon, Eli. Hammelseher, Sabine. Ondansetron in the treatment of Postoperative Vomiting. A & A, October 1992 vol 75. no. 4; 561-565.). One study determined that an intravenous dose of droperidol 0.625 mg administered at the start of anesthesia was as effective in preventing PONV following outpatient gynecological operations as intravenous droperidol 1.25 mg or ondansetron 4 mg. Droperidol caused no increase in sedation or other adverse effects. Similar anti-emetic effectiveness between intravenous ondansetron 4 mg and droperidol 20 μg/kg was shown when they were administered prior to anesthesia for outpatient gynaecological laparoscopy. There was no difference found in sedation between the droperidol and ondansetron groups. Fortney and co-workers reached similar conclusions. Fortney et al. conducted a multicentre, comparative study of intravenous ondansetron 4 mg, droperidol 0.625 mg or droperidol 1.25 mg versus placebo administered prior to induction of anesthesia with a barbiturate for PONV prevention. Droperidol 0.625 mg and 1.25 mg had similar anti-emetic effectiveness to ondansetron 4 mg. All anti-emetics evaluated were more effective than placebo (Fortney J T, Gan T J, Graczyk S, Wetchler B, Melson T, Khalil S, McKenzie R, Parrillo S, Glass P S A, Moote C, Wermeling D, Parasuraman T V, Duncan B, Creed M R, and the S3A-409 and S3A-410 Study Groups. Anesth Analg. 1998; 86:731-8). Furthermore, Fortney et al. found that the best anti-emetic effectiveness was obtained with the droperidol 1.25 mg dose, and this dose caused no increase in sedation or other adverse effects. There were more patients who received intravenous droperidol 2.5 mg and had no emesis than patients receiving intravenous ondansetron 8 mg in a PONV prophylaxis study in women undergoing inpatient minor gynaecological surgery. However, use of the intravenous droperidol 2.5 mg dose was associated with more adverse effects (i.e. sedation, dizziness) than that seen in other studies using a droperidol dose less than 2.5 mg. Polati and colleagues found that intravenous ondansetron 4 mg had better efficacy than intravenous metoclopramide 10 mg or placebo for the treatment of PONV following gynecological laparoscopy. Other researchers have reached a similar conclusion that intravenous ondansetron 4 mg has increased anti-emetic effectiveness compared with metoclopramide 10 mg for the treatment of PONV. (Polati, E., A & A 1997 August; 850 (2); 395-399, ondanseteron versus metodopranide in treatment of PONV). A comparative PONV treatment study between ondansetron and dolasetron was conducted by Roberson et al. in 92 patients after ambulatory surgery. (Meyer, Roberson et al. Dolasetron versus Ondansetron for treatment of postoperative nausea & vomiting. A&A February 2005, Vol 100. no. 2: 373-377.) Intravenous dolasetron 12.5 mg was determined to have significantly better anti-emetic efficacy in the PACU than intravenous ondansetron 4 mg on the basis of a lower requirement for rescue drugs in the PACU and greater patient satisfaction. An intravenous PONV prevention study in children undergoing adenotonsillectomy determined that intravenous ondansetron 0.1 mg/kg was more effective in preventing vomiting than dimenhydrimate 0.5 mg/kg or placebo. However, special note was made by the authors of this study that use of anti-emetics (to prevent vomiting) may mask the presence of blood in the stomach (from bleeding at the surgical site) and should be appreciated when adenotonsillectomy is performed on an outpatient basis. Desilva et al. compared the prophylactic anti-emetic efficacy of intravenous ondansetron 4 mg, perphenazine 5 mg, metoclopramide 10 mg, droperidol 1.25 mg and placebo administered prior to induction of anesthesia in inpatients undergoing total abdominal hysterectomy. (Desilva P H, Darvish A H, McDonald S M, Cronin M K, Clark K. Anesth Analg. 1995 July; 81(1):139-43. The efficacy of prophylactic ondansetron, droperidol, perphenazine, and metoclopramide in the prevention of nausea and vomiting after major gynecologic surgery.) Ondansetron, droperidol and perphenazine were determined to have similar anti-emetic effectiveness, and all 3 were found to be significantly better than metoclopramide or placebo. A PONV prevention comparison study by Purhonen et al. evaluated intravenous tropisetron 5 mg, droperidol 1.25 mg and placebo given at the end of gynaecological surgery. (Purhonen S, Kauko M, Koski E M, Nuutinen L. Anesth Analg. 1997 March; 84(3):662-7. Comparison of tropisetron, droperidol, and saline in the prevention of postoperative nausea and vomiting after gynecologic surgery.) These researchers determined that tropisetron 5 mg effectively prevented vomiting, but not nausea and retching. Droperidol 1.25 mg failed to prevent any PONV symptoms and resulted in an increase in anxiety and drowsiness. In an intravenous PONV prevention study, Korttila et al. found that dolasetron 50 mg and ondansetron 4 mg administered prior to anesthesia induction had similar anti-emetic effectiveness and were significantly more effective than dolasetron 25 mg or placebo. (Korttila K T, Jokinen J D. J Clin Anesth. 2004 August; 16(5):364-70. Timing of administration of dolasetron affects dose necessary to prevent postoperative nausea and vomiting.) The protocol design of this study differed from another intravenous dolasetron PONV prevention study by Gracyzk et al., in which intravenous dolasetron 12.5 mg was found to be effective when administered 15 to 30 minutes before the end of surgery. (Graczyk S G, McKenzie R, Kallar S, Hickok C B, Melson T, Morrill B, Hahne W F, Brown R A. Anesth Analg. 1997 February; 84(2):325-30. Intravenous dolasetron for the prevention of postoperative nausea and vomiting after outpatient laparoscopic gynecologic surgery.) The administration timing schedule change was made to conform to the approved administration schedule of the comparator drug (i.e. ondansetron). Zarate et al. compared the cost effectiveness of dolasetron (12.5 or 25 mg) and ondansetron (4 or 8 mg) for prophylaxis of PONV after ambulatory surgery. Total costs were calculated using the perspective of a surgical centre (Zarate E, Watcha M F, White P F, Klein K W, Sa Rego M, Stewart D G. Anesth Analg. 2000 June; 90(6):1352-8. A comparison of the costs and efficacy of ondansetron versus dolasetron for antiemetic prophylaxis.). The total costs were lowest in the dolasetron 12.5 mg group. Excluding nursing labor costs did not change this finding. A study by Naguib and co-workers gives insight into comparisons among the 5-HT3 receptor antagonists (Naguib M., Can J. Anaesth. 1996 March; 43(3):226-31. Prophylactic antiemetic therapy with ondansetron, tropisetron, granisetron and metoclopramide in patients undergoing laparoscopic cholecystectomy: a randomized, double-blind comparison with placebo.) For a PONV prevention study, these researchers compared intravenous ondansetron 4 mg with granisetron 3 mg, tropisetron 5 mg, metoclopramide 10 mg or placebo. All doses were administered before the start of anesthesia. The 5-HT3 receptor antagonists (ondansetron, tropisetron, granisetron) had significantly greater anti-emetic effectiveness than metoclopramide or placebo. There was no statistical difference in efficacy between the ondansetron, tropisetron and granisetron groups. When administered immediately prior to anesthesia, intravenous granisetron 2.5 mg was determined to be more effective than droperidol 1.25 mg or metoclopramide 10 mg in preventing PONV after major gynecological surgery in female patients with a history of postoperative emesis and motion sickness. Similarly, a third study by Fujii et al., concluded that granisetron 40 μg/kg administered immediately prior to anesthesia induction was more effective than droperidol 25 μg/kg or metoclopramide 0.2 mg/kg for prevention of PONV after major gynecological surgery in women during menstruation. (Fujii Y, Tanaka H, Somekawa Y. Am J Obstet Gynecol. 2000 January; 182(1 Pt 1):13-6. Granisetron, droperidol, and metoclopramide for the treatment of established postoperative nausea and vomiting in women undergoing gynecologic surgery.) In summary, regarding intravenous PONV prevention, numerous studies have determined that droperidol has similar anti-emetic effectiveness to ondansetron. Ondansetron, granisetron, tropisetron and droperidol had significantly better PONV effectiveness than metoclopramide when administered before anesthesia. Regarding intravenous treatment for PONV, two studies determined that ondansetron had significantly better anti-emetic effectiveness than metoclopramide, while another study determined less effectiveness than dolasetron. However, a significantly higher percentage of patients getting single agents remained with persistent symptoms despite escalating the doses. Furthermore, escalating the dose of the agents resulted in much higher incidence of side effects specific to that agent.

Included for reference is a table which lists anti-emetic agents of different classes. See Table 3.

TABLE 3
Classes of Anti-Emetics Available

| Class | Specific Agents |
|---|---|
| Anicholinergics | Scopolamine |
| | Atropine |
| Dopamine Receptor Antagonists (D2) | Phenothiazine |
| | Benzamides |
| | Butryophenones |
| Antihistamines | Dimenhydrinate |
| | Diphenhydramine |
| | Cyclizine |
| | Hydroxyzine |
| Benzodiazepines | Lorazepam |
| | Dizaepam |
| | Midazolam |
| Serotonin Receptor Antagonists (5-HT3) | Ondansetron |
| | Granisetron |
| | Dolasetron |
| | Palonosetron |
| | Tropisetron |
| Corticosteroids | Dexamethasone |
| | Methylprednisone |
| Neurokinin Receptor Antagonist | Aprepitant |
| | Fosaprepitant - IV |
| Cannabinoids | Dronabinol |
| | Nabilone |

### Combination Anti-Emetic Regimens

### I—Post-Operative Nausea and Vomiting (PONV)

The many emetogenic neuroreceptors and neurochemicals in the midbrain suggest the effectiveness of anti-emetics will be increased by combining them, especially in the patient with severe and/or intractable symptoms. Combination anti-emetic therapy was a disadvantage with the older traditional anti-emetics (i.e. antihistamines, phenothiazines, butyrophenones, etc.) because of the possibility of additive toxic central nervous system (CNS) effects (i.e. hypotension, sedation, dry mouth, EPS, etc.). Recent studies have evaluated and supported the effectiveness of the 5-HT3 receptor antagonists when used in combination with other anti-emetics. McKenzie et al. conducted multiple combination anti-emetic studies. An initial study combined intravenous ondansetron 4 mg with intravenous dexamethasone 8 mg and concluded that the prevention of PONV was significantly greater in the combined ondansetron plus dexamethasone group compared with patients receiving ondansetron alone. (McKenzie R, Riley T J, Tantisira B, Hamilton D L. J Clin Anesth. 1997 February; 9(1):15-20. Effect of propofol for induction and ondansetron with or without dexamethasone for the prevention of nausea and vomiting after major gynecologic surgery.) In a follow-up study, the McKenzie research group found that the anti-emetic effectiveness for tubal banding operations of a prophylactic PONV combination of intravenous ondansetron 4 mg plus droperidol 1.25 mg given immediately prior to anesthesia was more effective than either ondansetron or droperidol alone. (McKenzie R, Uy N T, Riley T J, Hamilton D L. Anesth Analg. 1996 December; 83(6):1218-22. Erratum in: Anesth Analg 1997 March; 84(3):704. Droperidol/ondansetron combination controls nausea and vomiting after tubal banding.). Fujii, Tanaka and Toyooka combined granisetron with droperidol or dexamethasone and determined that anti-emetic effectiveness was improved over each anti-emetic alone. (Fujii Y, Toyooka H, Tanaka H. Anesth Analg. 1998 March; 86(3):613-6. Prevention of postoperative nausea and vomiting with a combination of granisetron and droperidol.) The combination of intravenous granisetron 2.5 mg plus droperidol 1.25 mg given immediately prior to induction of anesthesia for prevention of nausea and vomiting was found to be more effective than either granisetron or droperidol alone. In another combination study by these researchers on female patients receiving intravenous anti-emetics, the number of patients who had nausea or emesis or who required anti-emetic medication was significantly less when intravenous dexamethasone 8 mg was combined with intravenous granisetron 20 μg/kg, compared with patients who received dexamethasone or granisetron alone. (Fujii Y, Tanaka H, Toyooka H. Anesth Analg. 1997 October; 85(4):913-7. The effects of dexamethasone on antiemetics in female patients undergoing gynecologic surgery.) Another study published by this group researched the effects of either intravenous droperidol 1.25 mg, metoclopramide 10 mg, or granisetron 40 μg/kg alone or with each anti-emetic combined with dexamethasone 8 mg in female patients undergoing major gynaecological surgery. (Fujii Y, Tanaka H, Toyooka H. Acta Anaesthesiol Scand. 1998 September; 42(8):921-5. Prevention of nausea and vomiting with granisetron, droperidol and metoclopramide during and after spinal anaesthesia for caesarean section: a randomized, double-blind, placebo-controlled trial.) Dexamethasone improved the anti-emetic efficacy of granisetron but did not improve the anti-emetic effectiveness of the other medications. The effect of combination anti-emetic therapy also was studied during morphine patient-controlled analgesia (PCA). The PCA solution (ondansetron 4 mg intravenous bolus plus a 0.13 mg/ml ondansetron infusion combined with droperidol 1.25 mg intravenous bolus plus a 0.05 mg/ml droperidol infusion) was compared with intravenous ondansetron 4 mg or droperidol 1.25 mg after major gynecological surgery. (McKenzie R, Tantisira B, Jackson D, Bach T, Riley T. J Clin Anesth. 1995 March; 7(2):141-7. Antiemetic efficacy of a droperidol-morphine combination in patient-controlled analgesia.) Improved anti-emetic efficacy was attained with the ondansetron plus droperidol combination compared with either anti-emetic alone. Koivuranta and co-workers concluded that better prophylactic anti-emetic efficacy with a lower degree of sedation occurred with the combination of intravenous ondansetron 8 mg plus droperidol 0.75 mg compared with ondansetron 8 mg plus droperidol 1.25 mg given before anesthesia. (Koivuranta, M., Jokela, R., Kiviluoma, K. and Alahuhta, S. (1997), The anti-emetic efficacy of a combination of ondansetron and droperidol. Anaesthesia, 52: 863-868. doi: 10.1111/j.1365-2044.1997.234-az0366.x) Pueyo et al. studied the intravenous combination of ondansetron 4 mg and droperidol 2.5 mg at the time of anesthesia induction, plus intravenous droperidol 1.25 mg 12 hours later, in elective abdominal surgery. (Pueyo F J, Carrascosa F, Lopez L, Iribarren M J, García-Pedrajas F, Saez A. Anesth Analg. 1996 July; 83(1):117-22. Combination of ondansetron and droperidol in the prophylaxis of postoperative nausea and vomiting.) The combination of ondansetron plus droperidol was more effective than each anti-emetic alone or placebo. The combination of intravenous droperidol 0.625 mg plus metoclopramide 10 mg was more effective than intravenous ondansetron 4 mg following laparoscopic cholecystectomy in a study by Steinbrook and co-workers. (Steinbrook R A, Freiberger D, Gosnell J L, Brooks D C. Anesth Analg. 1996 November; 83(5):1081-3. Prophylactic antiemetics for laparoscopic cholecystectomy: ondansetron versus droperidol plus metoclopramide.) An intravenous combination prevention study compared either (i) ondansetron 4 mg; (ii) dexamethasone 8 mg; (iii) ondansetron 4 mg plus dexamethasone 8 mg; or (iv) placebo during major gynaecological surgery. The anti-emetic effect of ondansetron plus dexamethasone was more than in the other study groups. No difference was found between ondansetron and dexamethasone when they were administered alone. Dexamethasone, however, appeared to be more effective in preventing nausea than emesis. Overall, a majority of studies show that the combination of anti-emetic medications acting at different emetogenic receptor sites had significantly better effectiveness in preventing nausea/vomiting than a single anti-emetic acting at 1 receptor site alone. The multimodal management of PONV is an important concept that has been previously proposed and recently tested. Scuderi et al. investigated a predefined multimodal clinical care algorithm for the prevention of PONV following outpatient laparoscopy. (Scuderi P E, James R L, Harris L, Mims G R 3rd. Anesth Analg. 2000 December; 91(6):1408-14. Multimodal antiemetic management prevents early postoperative vomiting after outpatient laparoscopy.) Their multimodal management consisted of total intravenous anesthesia (propofol and remifentanil), no N₂O, no neuromuscular blockade, aggressive intravenous hydration (25 ml/kg), triple anti-emetic combination (ondansetron, droperidol, dexamethasone) and ketorolac. Multimodal management was determined to have superior efficacy in preventing symptomatic post-operative nausea and vomiting compared with routine monotherapy prophylaxis.

### II—Chemotherapy Induced Nausea and Vomiting

The primary action site of 5-HT3 receptor antagonists is at the abdominal vagal afferents. Cytotoxic agents and other toxins increase release of serotonin, which subsequently activates 5-HT3 receptors. This signal to the serotonin receptors associated with vagal afferents is abolished by 5-HT3 antagonists such as granisetron and ondansetron. Neurokinin NK1 receptor antagonists, on the other hand, have shown a broad spectrum of action treating diverse causes of nausea and vomiting. Neurokinin NK1 receptor antagonists are thought to work at the dorsal vagal complex in the medulla, inhibiting the response that results in gastric emptying. The major psychoactive component of marijuana, tetrahydrocannabinol, may work to control emesis by direct relaxation of the stomach fundus. A variety of other neurotransmitters are involved in nausea/vomiting and the emetic reflex including dopamine, acetylcholine, endorphins, gamma-aminobutyric acid and histamine. Many of the other agents used to control nausea/vomiting target the release of these substances, which then trigger the emetic reflex. However, other agents such as steroids have an unknown mechanism controlling nausea/vomiting, particularly in some clinical situations like patients on highly emetogenic cisplatin based chemotherapy benefit from steroids alone.

Current literature clearly demonstrates that single agent therapy for nausea and emesis is ineffective in the setting of both auto-as well as allogeneic stem cell transplantation. Barbounis et al. showed that ondansetron as a single agent was only effective in about 76% of the cases on day 1 of chemotherapy administration. Furthermore, there was a progressive decline in controlling vomiting and nausea during subsequent days of chemotherapy. (Barbounis V, Koumakis G, Vassilomanolakis M, Hatzichristou H, Tsousis S, Efremidis AP. Support Care Cancer. 1995 September; 3(5):301-6. A phase II study of ondansetron as antiemetic prophylaxis in patients receiving high-dose polychemotherapy and stem cell transplantation.) Another study looked at the efficacy of tropisetron, a newer 5-HT3, not available in the United States at present. (Barbounis V, Koumakis G, Hatzichristou H, Vassilomanolakis M, Tsoussis S, Efremidis A. Support Care Cancer. 1999 March; 7(2):79-83. The anti-emetic efficacy of tropisetron plus dexamethasone in patients treated with high-dose chemotherapy and stem cell transplantation). In the first of these studies, there was response shown in about 80% of patients. The second study used a combination of tropisetron and dexamethasone with slightly better control at 83%. However, despite some response only 35% were protected from vomiting during the entire treatment period. Similar results were found for a third 5-HT3 agent dolasetron mesylate. Only 65% of patients had some emesis control. Thus, these single-arm studies clearly indicate the need for regimens that provided better acute and delayed emesis control.

Even worse is delayed control of nausea and emesis following induction chemotherapy. For example, prior studies used combination of 5-HT3 agent in combination with dexamethasone and lorazepam resulted in over 90% relief on day 1 of the start of chemotherapy, by day 6 only 46-50% of the patients had complete control of vomiting, again supporting the need for agents with better control. These results prompted the use of using multiple agents, and early results using a combination of regimens like granisetron, oral prochlorperazine and oral dexamethasone have been more encouraging. Another possible combination studied is aprepitant with 5-HT3 agents that have been promising.

Nausea and vomiting in the clinical setting have a multifactorial etiology. The pathogenesis of these symptoms involves multiple different neurobiochemical pathways. Hence, anti-emetic drugs from any one class are frequently ineffective in adequate control. Lack of symptom control frequently results in dose escalation and significant increase in adverse effects associated with the drug. Evolving literature strongly supports the role of combining anti-emetic drugs from different classes. Currently, combination anti-emetic therapies commonly include the parenteral use of different agents. However, parenteral use of different agents obviously cannot be done following discharge. Additionally, previous studies (as discussed above) have shown that a large majority of patients undergoing chemotherapy or an intervention will have persistent nausea and vomiting after discharge. Use of separate, multiple drugs results in non-compliance and adds to the overall cost of treatment.

A table of common side effects associated with different anti-emetics is included (See Table 4).

TABLE 4
Common Adverse Effects Associated with Single Agents

| Class | Drug | Adverse Effect |
|---|---|---|
| Anticholingeric | Scopolamine | Sedation, dry mouth, |
| | | Restlessness, Central |
| | | Cholingergic Syndrome |
| Phenothiazines | Chlorpromazine, | Sedation, EPS, hypotension, |
| | Promethazine, | restlessness, |
| | Perphenazine, | Anticholinergic Syndrome |
| | Prochlorperazine | |
| Antihistamines | Cyclizine, | Sedation, dry mouth, |
| | Hydroxyzine, | Anticholinergic Syndrome, |
| | Diphenhydramine | Restlessness |
| Butyrophenones | Droperidol, | Sedation, EPS, |
| | Haloperidol | hypotension, restlessness, |
| | | Neuroleptic Malignant |
| | | Syndrome |
| Benzamides | Metoclopramide, | Sedation, EPS, restlessness, |
| | Domperidone, | tachycardia hypertension, |
| | Benzquinamide | cardiac arrhythmias |
| Corticosteroids | Betamethasone | Adrenal suppression, would |
| | | healing |
| Serotonin Receptor | Ondansetron, | Headache, dizziness |
| Antagonists | Granisetron, | |
| | Tropisetron, | |
| | Dolasetron | |

In one embodiment of the invention, the pharmaceutical composition comprises four different doses of ondansetron and promethazine in masses as set forth in the table below. Each dose is delivered to the patient through various delivery methods including a single pill form, liquid, and intravenous formulations. See Table 5.

TABLE 5

| | | Relative Mass of Ondansetron |
|---|---|---|
| Ondansetron | Promethazine | to Promethazine |
| 4 mg | 12.5 mg | 0.32 |
| 4 mg | 25 mg | 0.16 |
| 8 mg | 12.5 mg | 0.64 |
| 8 mg | 25 mg | 0.32 |

In another embodiment of the invention, the pharmaceutical composition comprises two different doses of granisetron and promethazine in masses as set forth in the table below. Each dose is delivered to the patient through various delivery methods including a single pill form, liquid, and intravenous formulations. See Table 6.

TABLE 6

| | | Relative Mass of Granisetron |
|---|---|---|
| Granisetron | Promethazine | to Promethazine |
| 1 mg | 12.5 mg | 0.08 |
| 1 mg | 25 mg | 0.04 |

In another embodiment of the invention, the pharmaceutical composition comprises four different doses of dolastetron and promethazine in masses as set forth in the table below. Each dose is delivered to the patient through various delivery methods including a single pill form, liquid, and intravenous formulations. See Table 7.

TABLE 7

| | | Relative Mass of Dolastetron |
|---|---|---|
| Dolastetron | Promethazine | to Promethazine |
| 50 mg | 12.5 mg | 4 |
| 50 mg | 25 mg | 2 |
| 100 mg | 12.5 mg | 8 |
| 100 mg | 25 mg | 4 |

In yet another embodiment of the invention, the pharmaceutical composition comprises four different doses of aprepitant and pomethazine in masses as set forth in the table below. Each dose is delivered to the patient through various delivery methods including a single pill form, liquid, and intravenous formulations. See Table 8.

TABLE 8

| | | Relative Mass of Aprepitant |
|---|---|---|
| Aprepitant | Promethazine | to Promethazine |
| 40 mg | 12.5 mg | 3.2 |
| 40 mg | 25 mg | 1.6 |
| 80 mg | 12.5 mg | 6.4 |
| 80 mg | 25 mg | 3.2 |

In yet another embodiment, the anti-emetic composition comprises a NK1 receptor antagonist and a serotonin receptor antagonist.

In yet another embodiment, the anti-emetic composition comprises a NK1 receptor antagonist and an anticholinergic.

In yet another embodiment, the anti-emetic composition comprises a serotonin receptor antagonist and an anticholinergic.

In yet another embodiment, the anti-emetic composition comprises a serotonin receptor antagonist and a butyrophenone.

In yet another embodiment, the anti-emetic composition comprises a NK1 receptor antagonist and a butyrophenone.

In yet another embodiment, the anti-emetic composition comprises a serotonin receptor antagonist and a butyrophenone.

In yet another embodiment, the anti-emetic composition comprises a serotonin receptor antagonist and metoclopromide.

In yet another embodiment, the anti-emetic compositions comprises a NK1 receptor antagonist and metoclopromide.

In yet another embodiment, the anti-emetic compositions comprises a serotonin receptor antagonist and an antihistamine.

In yet another embodiment, the anti-emetic compositions comprises a NK1 receptor antagonist and an antihistamine.

It will now be evident to those skilled in the art that there has been described herein, an improved anti-emetic substance. Although the invention hereof has been described by way of a preferred embodiment, it will be evident that other adaptations and modifications can be employed without departing from the spirit and scope thereof. The terms and expressions employed herein have been used as terms of description and not of limitation; and thus, there is no intent of excluding equivalents, but on the contrary it is intended to cover any and all equivalents that may be employed without departing from the spirit and scope of the invention.

What is claimed is:

1. A pharmaceutical composition for the relief of nausea, emesis, or symptoms associated therewith comprising a combination of a dopamine receptor antagonist and an anti-emetic of a different class.

2. The pharmaceutical composition according to claim 1 wherein the dopamine receptor antagonist is a phenothiazine.

3. The pharmaceutical composition according to claim 2 wherein the phenothiazine is promethazine.

4. The pharmaceutical composition according to claim 3 wherein the anti-emetic of a different class is a serotonin receptor antagonist.

5. The pharmaceutical composition according to claim 4 wherein the serotonin receptor antagonist is selected from the group consisting of ondansetron, granisetron hydrochloride, or dolastetron.

6. The pharmaceutical composition according to claim 5 wherein the anti-emetic of a different class is a neurokinin receptor antagonist.

7. The pharmaceutical composition according to claim 6 wherein the neurokinin receptor antagonist is aprepitant.

8. The pharmaceutical composition according to claim 7 wherein the serotonin receptor antagonist is ondansetron and comprises at least three dosages in relative masses of ondansetron to promethazine that are about 0.16:1, 0.32:1 and 0.64:1.

9. The pharmaceutical composition according to claim 5 wherein the serotonin receptor antagonist is granisetron hydrochloride and comprises at least two, dosages in relative masses of granisetron hydrochloride to promethazine that are about 0.08:1 and 0.04:1.

10. The pharmaceutical composition according to claim 5 wherein the serotonin receptor antagonist is dolastetron and comprises at least three dosages in relative masses of dolastetron to promethazine that are about 2:1, 4:1, and 8:1.

11. The pharmaceutical composition according to claim 7 wherein the composition comprises at least three dosages in relative masses of aprepitant to promethazine that are about 1.6:1, 3.2:1, and 6.4:1.

12. A pharmaceutical composition for the relief of nausea, emesis, or symptoms associated therewith comprising a combination of a dopamine receptor antagonist and a neurokinin receptor antagonist.

13. The pharmaceutical composition according to claim 12 wherein the dopamine receptor antagonist is a phenothiazine.

14. A pharmaceutical composition for the relief of nausea, emesis, or symptoms associated therewith comprising a combination of a serotonin receptor antagonist and an anti-emetic agent of a different class; wherein the anti-emetic agent of a different class is selected from the group consisting of an anticholinergic, a neurokinin receptor antagonist, a butyrophenone, a metoclopromide, and an antihistamine.

15. A pharmaceutical composition for the relief of nausea, emesis, or symptoms associated therewith comprising a combination of a neurokinin receptor antagonist and an anti-emetic agent of a different class; wherein the anti-emetic agent of a different class is selected from the group consisting of an anticholinergic, a butyrophenone, a metoclopromide, and an antihistamine.
